(19) United States
(12) Patent Application Publication
(10) Pub. No.: US 2013/0115209 A1
(43) Pub. Date: May 9, 2013

(54) PROTECTIVE VACCINE BASED ON STAPHYLOCOCCUS AUREUS PROTEIN SA2412

(71) Applicants: Tessie B. McNeely, Gwynedd Valley, PA (US); Sharon B. Smith, Downingtown, PA (US); Julie K. Zorman, Quakertown, PA (US)

(72) Inventors: Tessie B. McNeely, Gwynedd Valley, PA (US); Sharon B. Smith, Downingtown, PA (US); Julie K. Zorman, Quakertown, PA (US)

(21) Appl. No.: 13/810,130

(22) PCT Filed: Jul. 11, 2011
(86) PCT No.: PCT/US11/43499

§ 371 Date: Jan. 14, 2013

Related U.S. Application Data
(60) Provisional application No. 61/364,477, filed on Jul. 15, 2010.

(51) Int. Cl. C07K 14/195; C07K 16/12

(52) U.S. Cl. CPC C07K 14/195; C07K 16/1271
USPC 424/133.1; 424/190.1; 424/139.1

(57) ABSTRACT

The present invention relates to methods of inducing an immune response to Staphylococcus comprising administering a composition comprising an SA2412 polypeptide from Staphylococcus aureus as well as derivatives or fragments thereof. The present also encompasses methods of treating and/or reducing the likelihood of a Staphylococcus infection by administering a composition comprising an antibody that specifically binds to an SA2412 polypeptide, derivative or fragments thereof. Compositions administered in the methods of the invention can include one or more additional antigens including, but not limited to, IsdB. Compositions used to practice the methods of the invention are also encompassed.

### FIELD OF INVENTION

The present invention relates to methods of inducing an immune response to Staphylococcus using an SA2412 protein from Staphylococcus aureus as well as derivatives or fragments thereof. The present invention also relates to a composition, particularly an S. aureus vaccine, comprising an SA2412 polypeptide, derivative or fragment thereof, alone or in combination with one or more additional immunogens, capable of inducing a protective immune response.

### BACKGROUND OF THE INVENTION

Staphylococcus aureus is a nosocomial as well as a community-acquired pathogen which causes several diseases ranging from minor skin infections to serious life-threatening wound infections, bacteraemia, endocarditis, pneumonia, osteomyelitis and toxic shock syndrome. See Lowy et al., 1998, N. Engl. J. Med. 339:520-32. The worldwide growing incidence of staphylococcal infections is strongly related to the increased use of surgical devices and a growing number of immunocompromised patients. The situation has become more serious since the increased use of antibiotics led to the emergence of methicillin-resistant S. aureus strains (MRSA). See Selvey et al., 2000, Infect. Control. Hosp. Epidemiol. 21:645-8; Peacock et al., 1980, Ann. Intern. Med. 93:526-32. Additionally, S. aureus isolates with reduced susceptibility to vancomycin, the antibiotic of choice against MRSA strains, were described in the lab as well as the clinic. See Tenover et al., 2001, Emerg. Infect. Dis. 7:327-32; Tenover et al., 1998, J. Clin. Microbiol. 36:1020-7; Palazzo et al., 2005, J. Clin. Microbiol. 43:179-85. The rising emergence of multidrug-resistant staphylococci has led to a growing interest in the development of alternative approaches to prevent and treat staphylococcal infections.

Information concerning S. aureus polypeptide sequences has been obtained from sequencing the S. aureus genome. See Kuroda et al., 2001, Lancet 357:1225-1240; Baba et al., 2000, Lancet 359:1819-1827; Gill et al., 2005, J. Bacteriol. 187:2426-2438 and European Patent Publication EP 0 786 519. To some extent, bioinformatics has been employed in efforts to characterize polypeptide sequences obtained from genome sequencing. See, e.g., European Patent Publication EP 0 786 519 and U.S. Pat. No. 6,593,114.

Techniques such as those involving display technology and sera from infected patients have also been used in an effort to help identify genes coding for potential antigens. See, e.g., International Publication Nos. WO 01/98499 and WO 02/059148; and Etz et al., 2002, Proc. Natl. Acad. Sci. USA 99:6573-6578. Numerous staphylococcal surface proteins have been identified so far using recently adopted technologies, like proteomics (see Brady et al., 2006, Infect. Immun. 74:3415; Gatlin et al., 2006, Proteomics 6:1530; Pieper et al., 2006, Proteomics 6:4246; Vytvytska et al., 2002, Proteomics 2:580; Nandakumar et al., 2005, J. Proteome Res. 4:250) or protein selection methods based on expression libraries (see Clarke et al., 2006, J. Infect. Dis. 193:1098; Etz et al., 2002, Proc. Natl. Acad. Sci. USA 99:6573-8; Weichhart et al., 2003, Infect. Immun. 71:4633; and Yang et al., 2006, Vaccine 24:1117).

Vaccines consisting of one or more antigenic determinants provide protection against lethal challenge with S. aureus in mice. See Stranger-Jones et al., 2006, Proc. Natl. Acad. Sci. USA 103:16942-7 and Kuklin et al., 2006, Infect. Immun. 74:2215. The SA2412 S. aureus protein is an ABC transporter protein (Gill et al., 2005, J. Bacteriol. 187:2426-2438). The ABC transporter family are diverse transmembrane proteins that utilize the energy of ATP hydrolysis to carry out certain biological processes including translocation of various substrates across membranes. The common feature of all ABC transporters is that they consist of two distinct domains, the transmembrane domain (TMD) and the nucleotide-binding domain (NBD) (Higgins, 1992, Annu. Rev. Cell Biol. 8:67).

The references cited in the present application are not admitted to be prior art to the claimed invention.

### SUMMARY OF THE INVENTION

The present invention is directed to compositions and methods for treating and/or decreasing the likelihood of an infection by Staphylococcus or a pathology associated with such an infection in a patient. Methods of the invention comprise administering to an animal or human patient in need thereof a composition comprising an SA2412 polypeptide, derivative or fragment thereof to generate a protective immune response. In another embodiment, methods of the invention comprise administering an antibody that specifically binds SA2412 to impart passive immunity to a patient in need thereof.

Accordingly, in one aspect of the invention, there is provided a composition comprising an isolated SA2412 polypeptide, derivative or fragment thereof and a pharmaceutically acceptable carrier. Preferably, said composition is a pharmaceutical composition such as a vaccine.

In a specific embodiment of the invention, one or more additional antigens are provided in the composition comprising an isolated SA2412 polypeptide, derivative or fragment thereof. The additional antigens that may be present include one or more additional S. aureus immunogens, one or more immunogens targeting other Staphylococcus organisms and/or one or more immunogens targeting other infectious organisms such as bacteria associated with nosocomial infections. In a preferred embodiment, the additional antigen is IsdB (also known as ORF0657) or a derivative or fragment thereof.

In another aspect of the invention, there is provided a composition comprising an antibody that specifically binds to SA2412. Preferably, the antibody is a human or humanized monoclonal antibody.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIGS. 1A-1B illustrate the sequence of SA2412 from S. aureus strain COL. The (A) amino acid sequence is SEQ ID NO:1 and (B) nucleotide sequence is SEQ ID NO:2.

FIG. 2 illustrates the amino acid sequence of IsdB from S. aureus strain COL (SEQ ID NO:3).

FIGS. 3A-3B depict the results from two challenge experiments using a rat indwelling catheter model. Rats were immunized with either SEQ ID NO:1 adsorbed onto amorphous aluminum hydroxyphosphate sulfate adjuvant (SA2412) or adjuvant alone and then challenged with S. aureus via the tail vein. Catheters were removed and evaluated for S. aureus colonization after 24 hours. The threshold at which catheter colonization was considered positive is shown (dotted line).

FIG. 4 depicts the results from a murine challenge experiment. Mice were immunized with SA2412 or BSA (negative control) and then challenged with a lethal does of S. aureus Becker via the tail vein. Survival was monitored for 10 days post challenge.

FIG. 5 shows the results from three independent murine lethal challenge experiments, as described in Example 4. In the first two experiments (see Panels A and B), mice were immunized with either (a) His-tagged SA2412 adsorbed onto AAHSA, or (b) BSA. Survival curves for the mice in Experiment #1 are depicted in FIG. 4. In the third experiment (panel C), mice immunized with His-tagged SA2412 adsorbed onto AAHSA were compared to naive (unimmunized) mice.

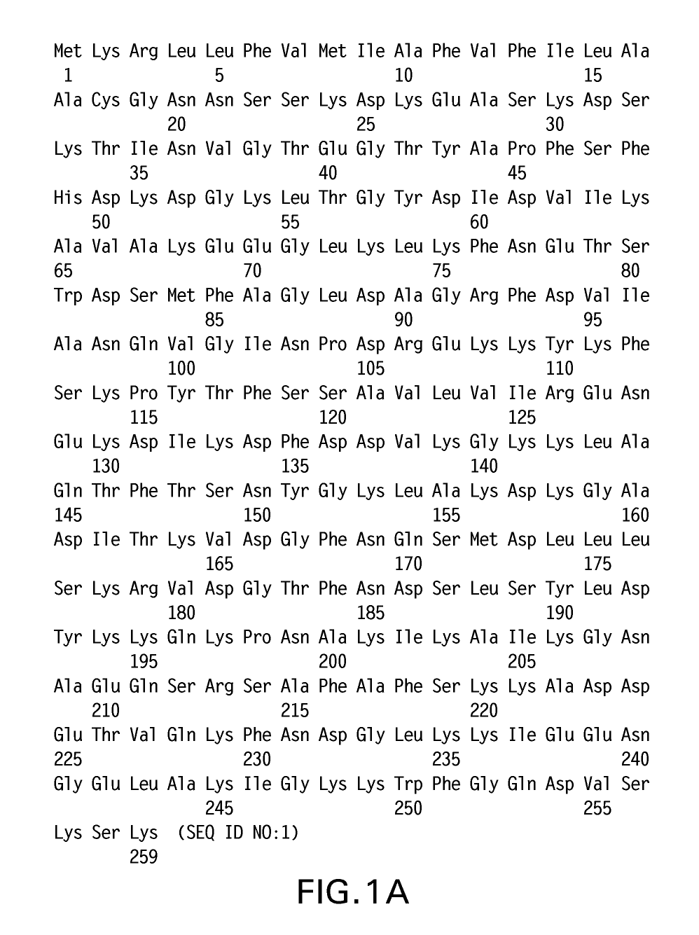

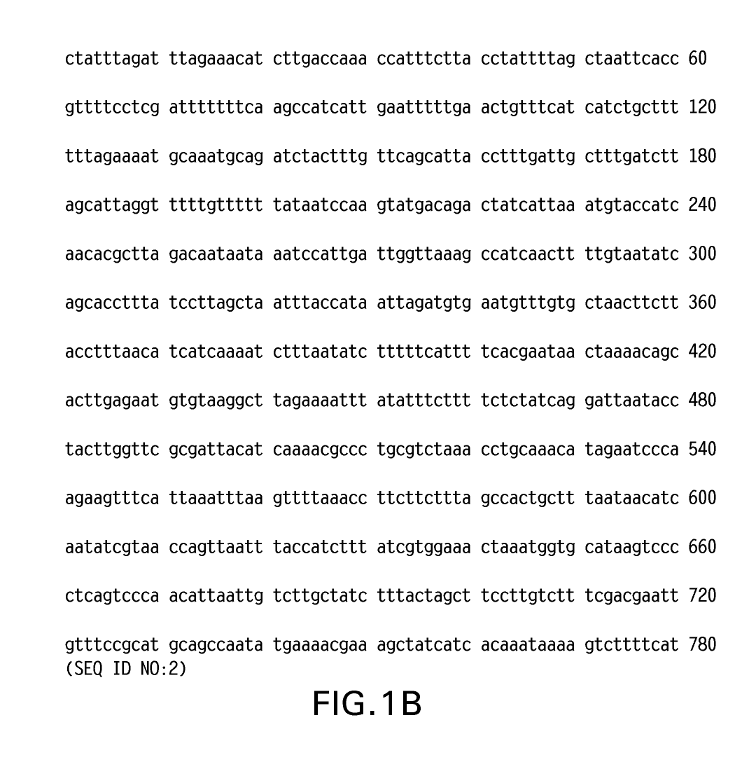

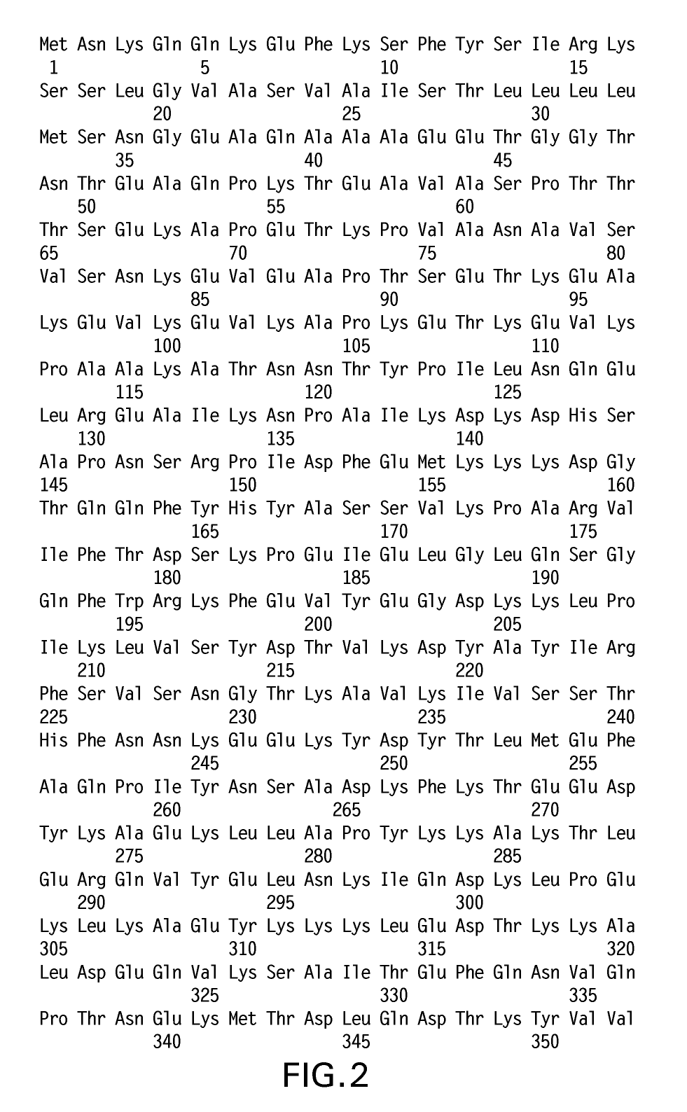

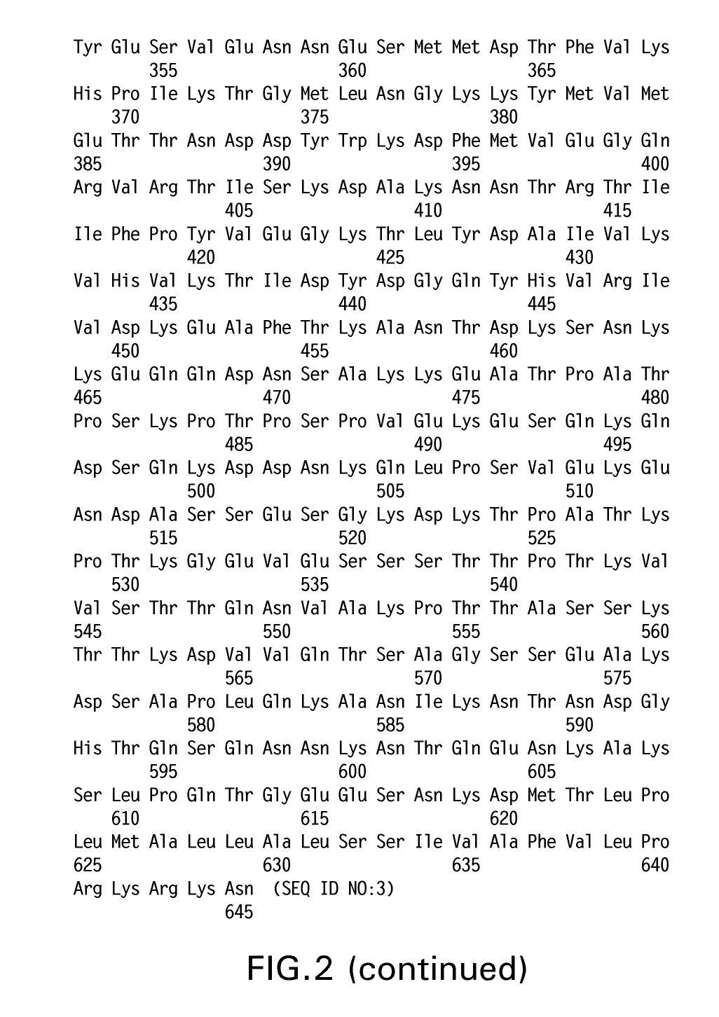

### DETAILED DESCRIPTION OF THE INVENTION

A S. aureus SA2412 protein was cloned and expressed recombinantly in E. coli. The recombinant protein is immunogenic in rodents and protects the animals from S. aureus infection. As used herein, the term “SA2412” refers to a polypeptide of SEQ ID NO:1 or a naturally occurring allelic variant or a homolog from another S. aureus strain. Examples of other strains of S. aureus include Becker, MW2, N315 (see Kuroda et al., 2001, Lancet 357:1225), Newman, USA300 (see Diep et al., 2006, Lancet 367:731), MSA817, and Mu3. In one embodiment, SA2412 is SEQ ID NO:1.

In one embodiment, SA2412 polypeptides, derivatives or fragments thereof are used as a vaccine for the treatment and/or reducing the likelihood of staphylococcal infections. Methods of the invention encompass administering a composition comprising a vaccine of the invention to a non-human animal or human patient in need thereof to induce an immune response. In a specific embodiment of the invention, one or more additional antigens are provided in the composition comprising an isolated SA2412 polypeptide, derivative or fragment thereof. In a preferred embodiment, the additional antigen is IsdB (also known as ORF0657) or a derivative or fragment thereof.

As used herein, the phrase “induce an immune response” refers to the ability of a polypeptide, derivative, or fragment thereof to produce an immune response in a patient, preferably a mammal, to which it is administered, wherein the response includes, but is not limited to, the production of elements, such as antibodies, which specifically bind S. aureus or said polypeptide, derivative or fragment thereof. The immune response provides a protective effect against S. aureus infection, ameliorates at least one pathology associated with S. aureus infection and/or reduces the likelihood that a patient will contract an S. aureus infection. In a specific embodiment, the immune response induces opsonophagocytic activity of human neutrophils for S. aureus.

As used herein, the phrase “an immunologically effective amount” refers to the amount of an immunogen that can induce a protective immune response against S. aureus when administered to a patient. The amount should be sufficient to significantly reduce the likelihood or severity of an S. aureus infection. Animal models known in the art can be used to assess the protective effect of administration of immunogen. For example, a murine, lethal-challenge model (see, e.g., Thakker et al., 1998, Inf Immun 66:5183-5189; Fattom et al., 1996, Inf Immun 64:1659-1665) and a rat, indwelling-catheter, sub-lethal challenge model (see, e.g., Ulphani et al., 1999, Lab Animal Sc. 49:283-287; Baddour et al., 1992, J Inf Dis 165:749-53; Ebert et al., Human Vaccines 7(6): 1-9 (2011)) can be used.

In another embodiment, SA2412 polypeptides, derivatives or fragments thereof are used as a target for generating antibodies. These antibodies can be administered to a patient for the treatment and/or reduction of the likelihood of staphylococcal infections due to passive immunity.

As used herein, the phrase “passive immunity” refers to the transfer of active humoral immunity in the form of antibodies. Passive immunity provides immediate protective effect to the patient from the pathogen recognized by the administered antibodies and/or ameliorates at least one pathology associated with pathogen infection. However, the patient does not develop an immunological memory to the pathogen and therefore must continue to receive the administered antibodies for protection from the pathogen to persist.

Embodiments also include one or more of the polypeptide immunogens or compositions thereof, described herein, or a vaccine comprising or consisting of said immunogens or compositions (i) for use in, (ii) for use as a medicament for, or (iii) for use in the preparation of a medicament for: (a) therapy (e.g., of the human body); (b) medicine; (c) inhibition of S. aureus replication; (d) treatment or prophylaxis of infection by S. aureus; or, (e) treatment, prophylaxis of, or delay in the onset or progression of S. aureus-associated disease(s). In these uses, the polypeptide immunogens, compositions thereof, and/or vaccines comprising or consisting of said immunogens or compositions can optionally be employed in combination with one or more anti-bacterial agents (e.g., anti-bacterial compounds; combination vaccines, described infra).

### Polypeptides

The amino acid sequence of a wild type full length SA2412 from S. aureus subsp. aureus COL is SEQ ID NO:1. SEQ ID NO:1 as well as derivatives and fragments thereof can be used in the methods of the invention. Collectively, derivatives and fragments of SEQ ID NO:1 are termed “altered polypeptides”.

As used herein, the term “isolated” indicates a different form than found in nature. The different form of the polypeptide can be, for example, a different purity than found in nature. In one embodiment, the term refers to polypeptides that are substantially or essentially free from components that normally accompany it in its native state.

As used herein, the terms “purified” with regard to, for example, a polypeptide immunogen indicates the presence of such polypeptide in an environment lacking one or more other polypeptides with which it is naturally associated and/or is represented by at least about 10% of the total protein present. In different embodiments, the purified polypeptide represents at least about 50%, at least about 75%, at least about 95%, or at least about 99% of the total protein in a sample or preparation.

As used herein, the term “fragment” refers to a continuous segment of an SA2412 polypeptide (i.e., SEQ ID NO:1) or derivatives thereof having at least 5 amino acid residues, at least 10 amino acid residues, at least 15 amino acid residues, or at least 20 amino acid residues and which is shorter than the full length SA2412 polypeptide. Preferably, fragments will comprise at least one antigenic determinant or epitopic region. In some embodiments, a fragment of the invention will comprise a domain of the SA2412 polypeptide including, but not limited to, the extracellular domain or T cell epitopes (either from the intracellular or extracellular portion of SA2412). One or more fragments comprising at least one antigenic determinant or epitopic region may be fused together.

As used herein, the terms “epitope” or “antigenic determinant” refer to a site on an antigen to which an antibody and/or T cell receptor binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance.

As used herein, the term “derivative” refers to a polypeptide having one or more alterations, which can be changes in the amino acid sequence (including additions and deletions of amino acid residues) and/or chemical modifications. In preferred embodiments, the derivative is at least 85%, at least 90%, at least 95%, at least 97%, at least 99% identical to the original sequence prior to alteration. In general, derivatives retain the activity of inducing a protective immune response. In some embodiments, SA2412 or a fragment thereof has been altered to a derivative of the invention such that one or more epitopes have been enhanced. Epitope enhancement improves the efficacy of a polypeptide to induce a protective immune response. Epitope enhancement can be performed using different techniques such as those involving alteration of anchor residues to improve peptide affinity for MHC molecules and those that increase the affinity of the peptide-MHC complex for a T-cell receptor (Berzofsky et al., 2001, Nature Review 1:209-219).

In one embodiment, a derivative is a polypeptide that has an amino acid sequence which differs from the base sequence from which it is derived by one or more amino acid substitutions. Amino acid substitutions may be regarded as “conservative” where an amino is replaced with a different amino acid with broadly similar properties. “Non-conservative” substitutions are where amino acids are replaced with amino acids of a different type. Broadly speaking, fewer non-conservative substitutions will be possible without altering the biological activity of the polypeptide. In some embodiments, no more than 12 amino acid residues, 11 amino acid residues, 10 amino acid residues, 9 amino acid residues, 8 amino acid residues, 7 amino acid residues, 6 amino acid residues, 5 amino acid residues, 4 amino acid residues, 3 amino acid residues, 2 amino acid residues, or 1 amino acid residue is/are substituted.

In another embodiment, a derivative is a polypeptide that has an amino acid sequence which differs from the base sequence from which it is derived by having one or more amino acid deletions and/or additions in any combination. Deleted or added amino acids can be either contiguous or individual residues. In some embodiments, no more than 25 amino acid residues, no more than 20 amino acid residues, no more than 15 amino acid residues, no more than 12 amino acid residues, no more than 10 amino acid residues, no more than 8 amino acid residues, no more than 7 amino acid residues, no more than 6 amino acid residues, no more than 5 amino acid residues, no more than 4 amino acid residues, no more than 3 amino acid residues, no more than 2 amino acid residues, or no more than 1 amino acid residue is/are deleted or added. In additional embodiments, domains of SA2412 including, but not limited to, the transmembrane domain, are deleted. Addition of amino acids may include fusion (either directly or via a linker) to at least one functional protein domain including, but not limited to, marker polypeptides, carrier polypeptides (including, but not limited to, OMPC, BSA, OVA, THY, KLH, tetanus toxoid, HbSAg, HBcAg, rotavirus capsid proteins, L1 protein of the human papilloma virus, diptheria toxoid CRM197 protein, flagellin and HPV VLP especially type 6, 11 and 16), polypeptides holding adjuvant properties or polypeptides that assist in purification. Additionally, it will be appreciated that the additional amino acid residues can be derived from S. aureus or an unrelated source and may produce an immune response effective against S. aureus or another pathogen.

In another embodiment, a derivative is a polypeptide that has an amino acid sequence which differs from the base sequence from which it is derived by having one or more chemical modifications of the protein. Chemical modifications include, but are not limited to, modification of functional groups (such as alkylation, hydroxylation, phosphatation, thiolation, carboxilation and the like), incorporation of unnatural amino acids and/or their derivatives during protein synthesis and the use of crosslinkers and other methods which impose conformational constraints on the polypeptides.

Any method known in the art can be used to determine the degree of difference between SA2412 (e.g., SEQ ID NO:1) and a derivative. In one embodiment, sequence identity is used to determine relatedness. Derivatives of the invention will be at least 85% identical, at least 90% identical, at least 95% identical, at least 97% identical, at least 99% identical to the base sequence (e.g., SEQ ID NO:1). The percent identity is defined as the number of identical residues divided by the total number of residues and multiplied by 100. If sequences in the alignment are of different lengths (due to gaps or extensions), the length of the longest sequence will be used in the calculation, representing the value for total length.

In another embodiment, hybridization is used to determine relatedness. Nucleic acids encoding derivatives of the invention will hybridize to nucleic acids encoding SA2412 (e.g., SEQ ID NO:2) under highly stringent conditions. Stringency of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3; and the temperature is at least about 30° C. for short probes, primers or oligonucleotides (e.g., 10 to 50 nucleotides) and at least about 60° C. for longer probes, primers and oligonucleotides. Stringent conditions may also be achieved with the addition of destabilizing agents, such as formamide. For additional details and explanation of stringency of hybridization reactions, see Ausubel et al., Current Protocols in Molecular Biology, Wiley Interscience Publishers, (1995).

As used here, the phrase “high stringency” refers to conditions that: (1) employ low ionic strength and high temperature for washing, for example 0.015 M sodium chloride/0.0015 M sodium citrate, 0.1% SDS at 50° C.; (2) employ a denaturing agent, such as formamide, during hybridization for example, 50% (v/v) formamide with 0.1% BSA, 0.1% Ficoll, 0.1% polyvinylpyrrolidone, 50 mM sodium phosphate buffer at pH 6.5 with 750 mM sodium chloride/50 mM sodium citrate at 42° C.; or (3) employ 50% formamide, 5×SSC, 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2.×SSC and at 55° C. in 50% formamide, followed by a high-stringency wash consisting of 0.1×SSC containing EDTA at 55° C.

### Polypeptide Production

The polypeptides, derivatives or fragments thereof for use in the methods of the invention can be produced recombinantly and, if needed, chemically modified. Recombinant expression of a polypeptide requires construction of an expression vector containing a polynucleotide that encodes the polypeptide of interest (i.e., an SA2412 polypeptide, derivative or fragment thereof). Once a polynucleotide encoding the polypeptide of interest has been obtained, the vector for the production of the polypeptide of interest may be produced by recombinant DNA technology using techniques well known in the art. Thus, methods for preparing a polypeptide of interest by expressing a polynucleotide encoding said polypeptide are described herein.

Methods which are well known to those skilled in the art can be used to construct expression vectors containing the coding sequences of the polypeptide of interest and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a polypeptide of interest operably linked to a promoter.

The expression vector is transferred to a host cell by conventional techniques and the transfected cells are then cultured by conventional techniques to produce the polypeptide of interest. Thus, the invention includes host cells containing a polynucleotide encoding a polypeptide of interest operably linked to a heterologous promoter.

A variety of host-expression vector systems may be utilized to express the polypeptides of interest. Such host-expression systems represent vehicles by which the coding sequences of interest may be produced and subsequently purified, but also represent cells which may, when transformed or transfected with the appropriate nucleotide coding sequences, express the polypeptide of interest in situ. These include but are not limited to microorganisms such as bacteria (e.g., E. coli, members of the Staphylococcus genus, such as S. aureus and S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens.) transformed with recombinant bacteriophage DNA, plasmid DNA or cosmid DNA expression vectors containing coding sequences of interest; yeast (e.g., Saccharomyces genus such as S. cerevisiae or S. pichia, members of the Pichia genus such as P. pastoris, members of the Hansenula genus such as H. polymorpha, members of the Kluyverornyces genus such as K. lactis or K. fragilis, and members of the Schizosaccharomyces genus such as S. pombe) transformed with recombinant yeast expression vectors containing coding sequences of interest; insect cell systems infected with recombinant virus expression vectors (e.g., baculovirus) containing coding sequences of interest; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences of interest; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter) and coding sequences of interest.

In addition, a host cell strain may be chosen which modulates the expression of the inserted sequences, or modifies and processes the gene product in the specific fashion desired. Eukaryotic modifications may include glycosylation and processing (e.g., cleavage) of protein products. Different host cells have characteristic and specific mechanisms for the post-translational processing and modification of proteins and gene products. Appropriate cell lines or host systems can be chosen to ensure the correct modification and processing of the foreign protein expressed. To this end, host cells which possess the cellular machinery for proper processing of the primary transcript, glycosylation, and phosphorylation of the gene product may be used. As used herein, the terms “polypeptide” or “an amino acid sequence of a polypeptide” includes polypeptides containing one or more amino acids having a structure of a post-translational modification from a host cell, such as a yeast host.

Once a polypeptide of interest has been produced by recombinant expression, it may be purified by different methods, for example, by chromatography (e.g., ion exchange, affinity, sizing column chromatography), centrifugation, differential solubility, or by any other standard technique for the purification of proteins. Further, the polypeptides of interest may be fused or attached to heterologous polypeptide sequences described herein or otherwise known in the art to facilitate purification. Examples of such protein tags include, but are not limited to, chitin binding protein (CBP), maltose binding protein (MBP), glutathione-S-transferase (GST), poly-histidine, hemagglutinin (HA) and polyanionic amino acids.

If desired, expression in a particular host can be enhanced through codon optimization. Codon optimization includes use of more preferred codons. Techniques for codon optimization in different hosts are well known in the art.

### Determination of Immunoreactive Derivatives and Fragments

The invention also extends to a method of identifying an immunoreactive derivative or fragments (collectively “altered polypeptides”) of an SA2412 polypeptide. This method essentially comprises generating a derivative or fragment of the polypeptide, administering the altered polypeptide to a mammal; and detecting an immune response in the mammal. Such response will include production of elements which specifically bind S. aureus and/or said polypeptide, derivative or fragment and/or have a protective effect against S. aureus infection. Antibody titers and immunoreactivity against the native or parent polypeptide may then be determined by, for example, radioimmunoassay, ELISA, western blot or ELISPOT.

### Adjuvants

Adjuvants are substances that can assist an immunogen (e.g., a polypeptide, pharmaceutical composition containing a polypeptide) in producing an immune response. Adjuvants can function by different mechanisms such as one or more of the following: increasing the antigen biologic or immunologic half-life; improving antigen delivery to antigen-presenting cells; improving antigen processing and presentation by antigen-presenting cells; and, inducing production of immunomodulatory cytokines (Vogel, Clinical Infectious Diseases 30(suppl. 3):S266-270, 2000). In one embodiment of the present invention, an adjuvant is used.

A variety of different types of adjuvants can be employed to assist in the production of an immune response. Examples of particular adjuvants include aluminum hydroxide; aluminum phosphate, aluminum hydroxyphosphate, amorphous aluminum hydroxyphosphate sulfate adjuvant (AAHSA) or other salts of aluminum; calcium phosphate; DNA CpG motifs; monophosphoryl lipid A; cholera toxin; E. coli heat-labile toxin; pertussis toxin; muramyl dipeptide; Freund's incomplete adjuvant; MF59; SAF; immunostimulatory complexes; liposomes; biodegradable microspheres; saponins; nonionic block copolymers; muramyl peptide analogues; polyphosphazene; synthetic polynucleotides; IFN-γ; IL-2; IL-12; and ISCOMS. (Vogel, Clinical Infectious Diseases 30(suppl 3):S266-270, 2000; Klein et al., 2000, Journal of Pharmaceutical Sciences 89:311-321; Rimmelzwaan et al., 2001, Vaccine 19:1180-1187; Kersten, 2003, Vaccine 21:915-920; O'Hagen, 2001, Curr. Drug Target Infect. Disord. 1:273-286.)

### Combination Vaccines

An SA2412 polypeptide, derivative or fragment thereof can be used alone, or in combination with other immunogens, to induce an immune response. Additional immunogens that may be present include one or more additional S. aureus immunogens, one or more immunogens targeting one or more other Staphylococcus organisms such as S. epidermidis, S. haemolyticus, S. warneri, S. pyogenes, or S. lugunensi, and/or one or more immunogens targeting other infectious organisms including, but not limited to, the pathogenic bacteria H. influenzae, M. catarrhalis, N. gonorrhoeae, E. coli, S. pneumoniae, C. difficile, C. perfringens, C. tetani, bacteria of the genuses Klebsiella, Serratia, Enterobacter, Proteus, Pseudomonas, Legionella, and Citrobacter.

In one embodiment, the additional immunogen is IsdB (also known as ORF0657) or related polypeptides. Reference to an IsdB immunogen refers to an immunogen that produces a protective immune response that recognizes the IsdB protein in S. aureus. In different embodiments, the IsdB immunogen produces an immune response that recognizes IsdB present on one or more of the following strains: COL, Becker, MW2, N315, Newman, USA300, MSA817, and Mu3. The ability of an IsdB immunogen to provided protective immunity is illustrated in, for example, US Publication No. 2006/0177462 (which is incorporated by reference herein in its entirety).

In additional embodiments, the IsdB immunogen comprises a polypeptide region, said region (a) is at least 90%, at least 94%, at least 95% or at least 99% identical to SEQ ID NO:3 or a fragment thereof (including, but not limited to, amino acids 42-486, 42-522 and 42-608 of SEQ ID NO:3); (b) differs from SEQ ID NO:3 or a fragment thereof (including, but not limited to, amino acids 42-486, 42-522 and 42-608 of SEQ ID NO:3) by 0, 1, 2, 3, 4, 5, 6, 7, 8, 9, 10, 11, 12, 13, 14, 15, 16, 17, 18, 19, 20, 21, 22, 23, 24, or 25 alterations, or up to 50 alterations; or (c) consists essentially or consists of SEQ ID NO:3 or a fragment thereof (including, but not limited to, amino acids 42-486, 42-522 and 42-608 of SEQ ID NO:3). Examples of alterations include amino acid substitutions, deletions, and insertions.

Reference to “consists essentially” of indicated amino acids indicates that the referred to amino acids are present and additional amino acids may be present. The additional amino acids can be at the carboxyl or amino terminus. In different embodiments 1, 2, 3, 4, 5, 6, 7, 8, 9, 10, 11, 12, 13, 14, 15, 16, 17, 18, 19, or 20 additional amino acids are present. In preferred embodiments methionine is present at the amino terminus; or methionine-glycine is present at the amino terminus.

In other embodiments, the one or more additional immunogens include, but are not limited to, ORF0657/ORF0190 hybrid polypeptides (International Publication No. WO 05/009378 and US Publication No. 2006/0188515); ORF0688-related polypeptides; ORF0452-related polypeptides (US Publication 2008/131447); ORF0912-related polypeptides; ORF1902-related polypeptides; sai-1-related polypeptides (International Publication No. WO 05/79315); ORF0594-related polypeptides (International Publication No. WO 05/086663); ORF0826-related polypeptides (International Publication No. WO 05/115113); PBP4-related polypeptides (International Publication No. WO 06/033918); AhpC-related polypeptides and AhpC-AhpF compositions (International Publication No. WO 06/078680); S. aureus type 5 and type 8 capsular polysaccharides (Shinefield et al., 2002, N. Eng. J. Med. 346:491-496); collagen adhesin, fibrinogen binding proteins, and clumping factor (Mamo et al., 1994, FEMS Immunol. Med. Microbiol. 10:47-54; Nilsson et al., 1998, J. Clin. Invest. 101:2640-2649; Josefsson et al., 2001, J. of Infect. Dis. 184:1572-1580); and polysaccharide intercellular adhesin and fragments thereof (Joyce et al., 2003, Carbohydrate Research 338:903-922).

### Nucleic Acid Vaccine

The nucleic acid sequence of wild type full length SA2412 from S. aureus subsp. aureus COL is SEQ ID NO:2. SEQ ID NO:2 or other nucleic acids that encode an SA2412 polypeptide of SEQ ID NO:1, derivative or fragment thereof can be introduced into a patient using vectors suitable for therapeutic administration. Suitable vectors can deliver the nucleic acid into a target cell without causing an unacceptable side effect. Examples of vectors that can be employed include plasmid vectors and viral based vectors. (Barouch, 2006, J. Pathol. 208:283-289; Emini et al., International Publication No. WO 03/031588.)

Cellular expression is achieved using a gene expression cassette encoding the desired polypeptide. The gene expression cassette contains regulatory elements for producing and processing a sufficient amount of nucleic acid inside a target cell to achieve a beneficial effect.

Examples of viral vectors include first and second generation adenovectors, helper dependent adenovectors, adeno-associated viral vectors, retroviral vectors, alphavirus vectors (e.g., Venezuelan Equine Encephalitis virus vectors), and plasmid vectors. (Hitt et al., 1997, Advances in Pharmacology 40:137-206; Johnston et al., U.S. Pat. No. 6,156,588; Johnston et al., International PCT Publication no. WO 95/32733; Barouch, 2006, J. Pathol. 208:283-289; Emini et al., International PCT Publication no. WO 03/031588.)

Adenovectors can be based on different adenovirus serotypes such as those found in humans or animals. Examples of animal adenoviruses include bovine, porcine, chimpanzee, murine, canine, and avian (CELO). (Emini et al., International PCT Publication no. WO 03/031588; Colloca et al., International PCT Publication no. WO 05/071093.) Human adenovirus include Group B, C, D, or E serotypes such as type 2 (“Ad2”), 4 (“Ad4”), 5 (“Ad5”), 6 (“Ad6”), 24 (“Ad24”), 26 (“Ad26”), 34 (“Ad34”) and 35 (“Ad35”).

Nucleic acid vaccines can be administered using different techniques and dosing regimes (see, e.g., International Publication No. WO 03/031588 and U.S. Pat. No. 7,008,791). For example, the vaccine can be administered intramuscular by injection with or without one or more electric pulses. Electric mediated transfer can assist genetic immunization by stimulating both humoral and cellular immune responses. Examples of dosing regimes include prime-boost and heterologous prime-boost approaches.

### SA2412 Antibodies

An SA2412 polypeptide, derivative or fragment thereof can be used to generate antibodies and antibody fragments that bind to SA2412 or to S. aureus. Such antibodies and antibody fragments can be used in polypeptide purification, S. aureus identification, and/ or in therapeutic treatment of S. aureus infection. In preferred embodiments, antibodies and/or antibody fragments thereof are administered to a patient in need thereof to provide passive immunity to S. aureus.

As used herein, the term “antibody” as used includes monoclonal antibodies, polyclonal antibodies, multispecific antibodies (e.g. bispecific antibodies), single-chain Fvs (scFv) (including bi-specific scFvs), single chain antibodies, Fab fragments, F(ab′) fragments, disulfide-linked Fvs (sdFv), and epitope-binding fragments of any of the above, so long as they exhibit the desired biological activity. In preferred embodiments, antibodies of the invention are monoclonal. In a more preferred embodiment, the monoclonal antibodies used in the methods of the invention are humanized or human antibodies.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which typically include different antibodies directed against different determinants (epitopes), each monoclonal antibody is directed against a single determinant on the antigen. The monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol 222: 581-597 (1991).

“Humanized” forms of non-human (e.g., murine) antibodies are immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. Thus, a humanized antibody is a recombinant protein in which the CDRs from an antibody from one species; e.g., a rodent antibody, is transferred from the heavy and light variable chains of the rodent antibody into human heavy and light variable domains. The constant domains of the antibody molecule are derived from those of a human antibody. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin (see, e.g., Yamashita et al., 2007, Cytotech. 55:55; Kipriyanov and Le Gall, 2004, Mol. Biotechnol. 26:39 and Gonzales et al., 2005, Tumour Biol. 26:31).

Completely human antibodies may be desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893 and WO 98/16654, each of which is incorporated herein by reference in its entirety. Human antibodies can also be produced using transgenic mice which are incapable of expressing functional endogenous immunoglobulins, but which can express human immunoglobulin genes., see, e.g., PCT publications WO 98/24893; European Patent No. 0 598 877; U.S. Pat. Nos. 5,916,771; and 5,939,598, which are incorporated by reference herein in their entireties.

In some embodiments, Fc engineered variants antibodies of the invention are also encompassed by the present invention. Such variants include antibodies or antigen binding fragments thereof which have been engineered so as to introduce mutations or substitutions in the Fe region of the antibody molecule so as to improve or modulate the effector functions of the underlying antibody molecule relative to the unmodified antibody. In general, improved effector functions refer to such activities as CDC, ADCC and antibody half life (see, e.g., U.S. Pat. Nos. 7,371,826; 7,217,797; 7,083,784; 7,317,091; and 5,624,821, each of which is incorporated herein in its entirety).

There are five classes of immunoglobulins: IgA, IgD, IgE, IgG, and IgM. The IgG and IgA classes are further divided into subclasses on the basis of relatively minor differences in the constant heavy region sequence and function, e.g., humans express the following subclasses: IgG1, IgG2, IgG3, IgG4, IgA1, and IgA2. In preferred embodiments, the antibodies of the invention are IgG1.

Proper glycosylation can be important for antibody function (Yoo et al., 2002, J. Immunol. Methods 261:1-20; Li et al., 2006, Nature Biotechnol. 24:210-215). Naturally occurring antibodies contain at least one N-linked carbohydrate attached to a heavy chain (Yoo et al., supra). Additional N-linked carbohydrates and O-linked carbohydrates may be present and may be important for antibody function Id.

Different types of host cells can be used to provide for efficient post-translational modifications including mammalian host cells and non-mammalian cells. Examples of mammalian host cells include Chinese hamster ovary (Cho), HeLa, C6, PC12, and myeloma cells (Yoo et al., supra; Persic et al., 1997, Gene 187:9-18). Non-mammalian cells can be modified to replicate human glycosylation (Li et al., 2006, Nature Biotechnol. 24:210-215). Glycoengineered Pichia pastoris is an example of such a modified non-mammalian cell (Li et al., supra).

### Patient Population

A “patient” refers to a mammal capable of being infected with S. aureus. In one preferred embodiment, the patient is a human. In alternative embodiments, the patient is a non-human mammal such as a dog. A patient can be treated prophylactically or therapeutically. Prophylactic treatment provides sufficient protective immunity to reduce the likelihood or severity of a S. aureus infection. Therapeutic treatment can be performed to reduce the severity of a S. aureus infection.

Prophylactic treatment can be performed using a pharmaceutical composition containing a polypeptide, immunogen or antibody described herein. Pharmaceutical compositions can be administered to the general population or to those persons at an increased risk of S. aureus infection.

Those “in need of treatment” include those already with an infection, as well as those prone to have an infection or in which a reduction in the likelihood of infection is desired. Persons with an increased risk of S. aureus infection include health care workers; hospital patients; patients with weakened immunity; patients facing therapy leading to a weakened immunity (e.g., undergoing chemotherapy or radiation therapy for cancer or taking immunosuppressive drugs); patients undergoing surgery; patients receiving foreign body implants (such a catheter or a vascular device); patients under diagnostic procedures involving foreign bodies; patients on renal dialysis and persons in professions having an increased risk of burn or wound injury. As used herein, “weakened immunity” refers to an immune system that is less capable of battling infections because of an immune response that is not properly functioning or is not functioning at the level of a normal healthy adult. Examples of patients with weakened immunity are patients that are infants, young children, elderly, pregnant or a patient with a disease that affects the function of the immune system such as HIV or AIDS.

Foreign bodies used in diagnostic or therapeutic procedures include indwelling catheters or implanted polymer device. Examples of foreign body-associated S. aureus infections include septicemia/endocarditis (e.g., intravascular catheters, vascular prostheses, pacemaker leads, defibrillator systems, prosthetic heart valves, and left ventricular assist devices); peritonitis (e.g., ventriculo-peritoneal cerebrospinal fluid (CSF) shunts and continuous ambulatory peritoneal dialysis catheter systems); ventriculitis (e.g., internal and external CSF shunts); and chronic polymer-associated syndromes (e.g., prosthetic joint/hip loosening, fibrous capsular contracture syndrome after mammary argumentation with silicone prosthesis and late-onset endophtalmisis after implantation of artificial intraocular lenses following cataract surgery). (See, Heilmann and Peters, Biology and Pathogenicity of Staphylococcus epidermidis, In: Gram Positive Pathogens, Eds. Fischetti et al., American Society for Microbiology, Washington D.C. 2000.)

Non-human patients that can be infected with S. aureus include cows, pigs, sheep, goats, rabbits, horses, dogs, cats, rats and mice. Treatment of non-human patients is useful in both protecting pets and livestock and evaluating the efficacy of a particular treatment.

In an embodiment, a patient is treated prophylactically in conjunction with a therapeutic or medical procedure involving a foreign body. In additional embodiments, the patient is immunized at about 2 weeks, 1 month, about 2 months or about 2-6 months prior to the procedure. In another embodiment, the patient is immunized prophylactically not in conjunction with a particular contemplated procedure. For vaccinations, boosters are delivered as needed. Additionally, patients treated prophylactically may also receive passive immunotherapy by administration of an antibody protective against S. aureus alone or in conjunction with vaccination.

### Pharmaceutical Compositions

A further feature of the invention is the use of an SA2412 polypeptide, derivative or fragment thereof described herein (“immunogenic agent”), either alone or in combination with one or more additional antigens, in a composition, preferably an immunogenic composition or vaccine, for treating patients with an S. aureus infection, reducing the progression, onset or severity of pathological symptoms associated with S. aureus infection and/or reducing the likelihood of an S. aureus infection. Suitably, the composition comprises a pharmaceutically acceptable carrier.

In some embodiment of the invention described above, the pharmaceutical compositions are used in human patients. In alternative embodiments, the pharmaceutical compositions are used in non-human patients.

A “pharmaceutically-acceptable carrier” is meant to mean a liquid filler, diluent or encapsulating substance that may be safely used in systemic administration. Depending upon the particular route of administration, a variety of pharmaceutically acceptable carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions including phosphate buffered saline, emulsifiers, isotonic saline, and pyrogen-free water. In particular, pharmaceutically acceptable carriers may contain different components such as a buffer, sterile water for injection, normal saline or phosphate-buffered saline, sucrose, histidine, salts and polysorbate. Terms such as “physiologically acceptable”, “diluent” or “excipient” can be used interchangeably.

The above compositions may be used as therapeutic or prophylactic vaccines. Accordingly, the invention extends to the production of vaccines containing as active ingredients one or more of the immunogenic agents of the invention. Any suitable procedure is contemplated for producing such vaccines. Exemplary procedures include, for example, those described in New Generation Vaccines (1997, Levine et al., Marcel Dekker, Inc. New York, Basel Hong Kong), which is incorporated herein by reference.

A polypeptide of the invention can be fused or attached to an immunogenic carrier. Useful carriers are well known in the art and include for example: thyroglobulin; albumins such as human serum albumin; toxins, toxoids or any mutant crossreactive material (CRM) of the toxin from tetanus, diptheria, pertussis, Pseudomonas, E. coli, Staphylococcus, and Streprococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a peptide of the invention can be coupled to a T cell epitope of a bacterial toxin, toxoid or CRM. In this regard, reference may be made to U.S. Pat. No. 5,785,973, which is incorporated herein by reference.

### Administration/Methods of Treatment

An SA2412 polypeptide, derivative or fragment thereof (alone or in combination with one or more immunogens) or an antibody described herein can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Accordingly, the invention provides a method for inducing a protective immune response in a patient against an S. aureus infection comprising the step of administering to the patient an immunologically effective amount of any of the vaccines or pharmaceutical compositions described herein. In one embodiment of this aspect of the invention, the patient is a human. In alternative embodiments, the patient is a non-human mammal.

Also provided by the invention is a method for treating S. aureus infection, or for treating any pathological condition associated with S. aureus infection, the method comprising the step of administering to the patient an immunologically effective amount of any of the vaccines or pharmaceutical compositions described herein. In one embodiment of this aspect of the invention, the patient is a human. In alternative embodiments, the patient is a non-human mammal.

Vaccines and/or antibodies can be administered by different routes such as subcutaneous, intramuscular, intravenous, mucosal, parenteral or transdermal. Subcutaneous and intramuscular administration can be performed using, for example, needles or jet-injectors.

In some embodiments, the vaccines and/or antibodies of the invention can be formulated in or on virus-like particles (see, e.g., International Publication Nos. WO94/20137,; WO96/11272; U.S. Pat. Nos. 5,985,610; 6,599,508; 6,361,778), liposomes (see, e.g., U.S. Pat. No. 5,709,879), bacterial or yeast ghosts (empty cells with intact envelopes; see, e.g., International Publication WO 92/01791, US Publication No. 2009/0239264 and 2008/0003239, U.S. Pat. No. 6,951,756), and outer membrane vesicles or blebs (de Moraes et al., 1992, Lancet 340: 1074 and Bjune et al., 1991, Lancet 338: 1093).

The compositions described herein may be administered in a manner compatible with the dosage formulation, and in such amount as is immunogenically-effective to treat and/or reduce the likelihood of S. aureus infection. The dose administered to a patient, in the context of the present invention, should be sufficient to effect a beneficial response in a patient over time such as a reduction in the level of S. aureus, or to reduce the likelihood of infection by S. aureus. The quantity of the immunogenic agent(s) to be administered may depend on the subject to be treated inclusive of the age, sex, weight and general health condition thereof. In this regard, precise amounts of the immunogenic agent(s) required to be administered will depend on the judgment of the practitioner. In determining the effective amount of the immunogenic agent to be administered in the treatment or prophylaxis against S. aureus, the physician may evaluate circulating plasma levels, progression of disease, and the production of anti-S. aureus antibodies. In any event, suitable dosages of the immunogenic agents of the invention may be readily determined by those of skill in the art. Such dosages may be in the order of nanograms to milligrams of the immunogenic agents of the invention.

Suitable dosing regimens are preferably determined taking into account factors well known in the art including age, weight, sex and medical condition of the patient; the route of administration; the desired effect; and the particular compound employed. The vaccine and/or antibody composition can be used in multi-dose formats. It is expected that a dose for a vaccine composition would consist of the range of 1.0 μg to 1.0 mg total polypeptide. In different embodiments of the present invention, the dosage range is from 5.0 μg to 500 μg, 0.01 mg to 1.0 mg, or 0.1 mg to 1.0 mg. When more than one polypeptide is to be administered (i.e., in combination vaccines), the amount of each polypeptide is within the described ranges.

It is expected that a dose for a passive immunity composition of the invention would consist of the range of 1 μg/kg to 100 mg/kg of antibody. In different embodiments, of the present invention, the dosage range is from 1 μg/kg to 15 mg/kg, 0.05 mg/kg to about 10 mg/kg, 0.5 mg/kg to 2.0 mg/kg, or 10 mg/kg to 50 mg/kg.

The timing of doses depends upon factors well known in the art. After the initial administration one or more additional doses may be administered to maintain and/or boost antibody titers.

For combination vaccinations, each of the polypeptides can be administered together in one composition or separately in different compositions. An SA2412 polypeptide described herein is administered concurrently with one or more desired immunogens. The term “concurrently” is not limited to the administration of the therapeutic agents at exactly the same time, but rather it is meant that the SA2412 polypeptides described herein and the other desired immunogen(s) are administered to a subject in a sequence and within a time interval such that the they can act together to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route.

### EXAMPLES

Examples are provided below to further illustrate different features of the present invention. The examples also illustrate useful methodology for practicing the invention. These examples do not limit the claimed invention.

### Example 1

### Production and Purification

E. coli competent cells were transformed with the vector containing the SA2412 ORF (SEQ ID NO:1) with a histidine tag and grown on LB plates containing ampicillin (100 μg/mL) and chloramphenicol (34 ng/ml). To test for expression of SA2412, an isolated colony was inoculated into 5 mL of liquid LB, 1% glucose, 100 μg/ml ampicillin, and incubated at 37° C. or 25° C., 250 rpm, until the OD₆₀₀ was between 0.5 to 1.0. A 1.0 ml culture volume of cells was subjected to centrifugation and resuspended in lysis buffer. The mixtures were held on ice for 5 minutes and subsequently sonicated three times for ten seconds, each with cooling in between. To obtain “soluble” and “insoluble” fractions the mixture was centrifuged at 13,000 rpm for fifteen minutes at 4° C. The supernatant was designated “soluble” and the pellet was resuspended in lysis buffer and designated “insoluble.”

Expression of His-tagged SA2412 was analyzed by Coomassie staining of SDS-PAGE gels run under reducing and denaturing conditions. The gels were stained with Bio-Safe Coomassie, a Coomassie G250 stain (BIO-RAD) according to the manufacturer's protocol. Western blot was performed and the signal was detected by anti-His mAb (EMD Sciences)

A 21.3 kDa protein was specifically detected by both Coomasie staining and Western blot in lysates. A 40 kDa protein was also detected, however, there was enough product of the correct size for purification. An increased amount of SA2412 (to about 80%) was observed in the soluble fraction when cultures were grown at 25° C. as compared to 37° C.

Direct scale-up of the above small scale procedure into stirred tank fermenters (30 liter scale) with a 20 liter working volume was achieved. Inoculum was cultivated in a 250 mL flask containing 50 mL of LB medium (plus ampicillin) and inoculated with 1 mL of frozen seed culture and cultivated for 6 hours. One mL of this seed was used to inoculate a 2 liter flask containing 500 mL of LB medium (plus ampicillin) and incubated for 16 hours. A large scale fermenter (30 liter scale) was cultivated with 20 liters of LB medium (plus ampicillin). The fermentation parameters of the fermenter were: pressure=5 psig, agitation speed=300 rpm, airflow=7.5 liters/minute and temperature=25° C. Cells were incubated to an optical density (OD) of 1.3 optical density units at a wavelength of 600 nm. Cells were harvested by lowering the temperature to 15° C., concentrated by passage through a 500 KMWCO hollow fiber cartridge, and centrifuged at 8,000 times gravity at 4° C. for 20 minutes. Supernatants were decanted and the recombinant E. coli wet cell pellets were frozen at −70° C.

Frozen recombinant E. coli cell paste (24 grams) was thawed and resuspended in two volumes of Lysis Buffer (50 mM sodium phosphate, pH 8.0, 0.15 M NaCl, 2 mM magnesium chloride, 10 mM imidazole, 20 mM 2-mercaptoethanol, 0.1% Tween-80, and protease inhibitor cocktail (Complete™, EDTA-Free, Roche #1873580-one tablet per 50 ml Lysis Buffer). Benzonase (EM #1.01697.0002) was added to the cell suspension at 125 Units/mL). A lysate was prepared with a microfluidizer. The lysate was stirred for three hours at 4° C., and was clarified by centrifugation at 10,000×g for 10 minutes at 4° C. The supernatant was filtered through a glass-fiber pre-filter Millipore and NaCl was added to a final concentration of 0.5 M from a 5 M stock solution. The Filtered Supernatant was added to Ni-NTA agarose chromatography resin (Qiagen #30250) and the slurry was mixed overnight at 4° C. The slurry of chromatography resin was poured into a chromatography column and the non-bound fraction was collected by gravity from the column outlet. The column was washed with ten column volumes of Wash Buffer (50 mM sodium phosphate, pH 8.0, 0.5 M NaCl, 2 mM magnesium chloride, 10 mM imidazole, 20 mM 2-mercaptoethanol, 0.1% Tween-80, and protease inhibitor cocktail (Complete™, EDTA-Free, Roche #1873580-one tablet per 50 ml Wash Buffer). The column was eluted with Elution Buffer (50 mM sodium phosphate, pH 7.4, 0.3 M imidazole, 2 mM magnesium chloride, 0.1% Tween-80, and 20 mM 2-mercaptoethanol). Fractions containing protein were identified by dot blot on nitrocellulose membrane with Ponceau-S staining, and fractions containing the highest protein concentrations were pooled to make the Ni-IMAC Product. The Ni-IMAC Product was fractionated by SEC. SEC fractions containing the product protein were identified by SDS/PAGE with Coomassie staining. Product-containing SEC fractions were pooled to make the SEC Product and was sterile-filtered. This protein was used in immunogenic and protection studies.

### Example 2

### Immunogenic Studies

Sprague-Dawley rats, 3-4 weeks of age, were immunized on Days 0, 7 and 21 intraperitoneally with a formulation of His-tagged SA2412 (20 μg per injection) adsorbed onto amorphous aluminum hydroxyphosphate sulfate adjuvant (AAHSA). Negative control rats were injected with adjuvant alone. The materials were administered as a single 100 μl intraperitoneal injection at each time point. The rats were bled on day 28 and their sera were screened for presence of an immune reaction to His-tagged SA2412 by two assays. Immune sera was analyzed by ELISA for reactivity to SA2412 as well as by flow cytometry for reactivity to S. aureus. Each assay revealed that the His-tagged SA2412 generated an immune response in the immunized rats. Negative control rats did not generate an immune response to SA2412 (data not shown).

In a similar study with Balb/c mice, mice at 3-5 weeks of age were immunized on days 0, 7 and 21 intramuscularly with a formulation of His-tagged SA2412 (20 μg per injection) adsorbed onto AAHSA. Negative control mice were injected with bovine serum albumin (BSA) formulated on AAHSA, or not immunized. Immune sera from the mice was tested for reactivity to SA2412 by ELISA and was found to contain high antibody titers to the antigen. Negative control antisera did not have antibody titers to SA2412 (Data not shown).

### Example 3

### Protection Studies Using a Rat Indwelling Catheter Model

A rat indwelling catheter model was used to assess whether active immunization against SA2412 can prevent S. aureus infection of implanted devices. Sprague-Dawley rats, 3-4 weeks of age, were immunized on Days 0, 7 and 21 intraperitoneally with a formulation of His-tagged SA2412 (20 μg per injection) adsorbed onto amorphous aluminum hydroxyphosphate sulfate adjuvant (AAHSA). Negative control rats were injected with adjuvant alone. The materials were administered as a single 100 μl intraperitoneal injection at each time point. On Day 35, the animals underwent surgery to place an indwelling catheter into the jugular vein. The animals were rested for approximately 10 days after surgery, at which time a sub-lethal challenge of S. aureus strain Becker (2.0×10⁹ CFU/rat) was given intravenously via the tail vein. The rats were sacrificed 24 hours post challenge and the catheters were removed using sterile procedures. The presence of S. aureus bacteria on the catheters was assessed by culturing the entire catheter in TSB containing 7.5% NaCl overnight using a cultivation system with a discontinuous optical density measurement (Piccolo® culture instrument, see Wollerton et al., 2006, J. Assoc. Lab. Auto. 11:292) to generate growth curves from catheter adherent S. aureus. Growth was monitored by absorbance at OD=600 nm by the Piccolo® instrument. CFUs on the catheters were estimated by comparison to growth curves generated by bacteria samples with known CFUs at time 0. Detection of greater than 1000 CFUs of S. aureus indicated that the catheter was effectively colonized and considered to lead to persistent infection. Catheters with les than 1000 CFUs were considered to not lead to persistent infection due to spontaneous clearance of the catheter by the rat immune system.

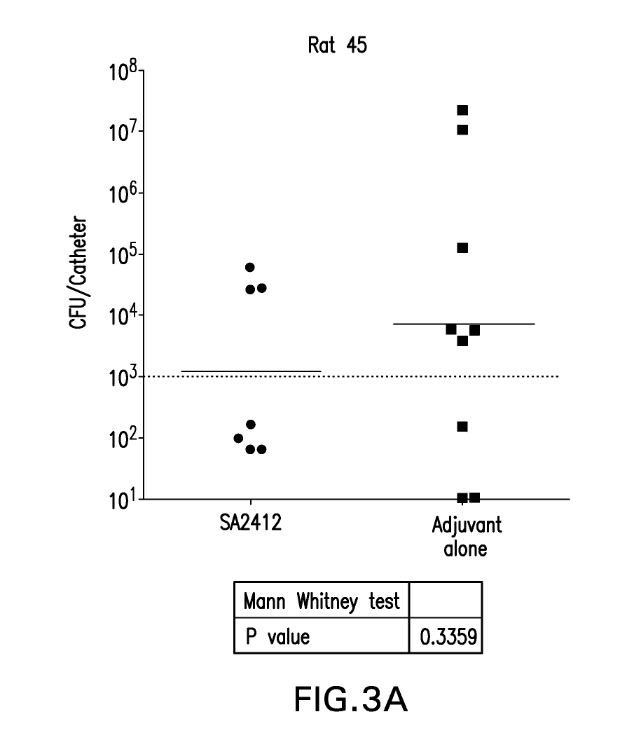

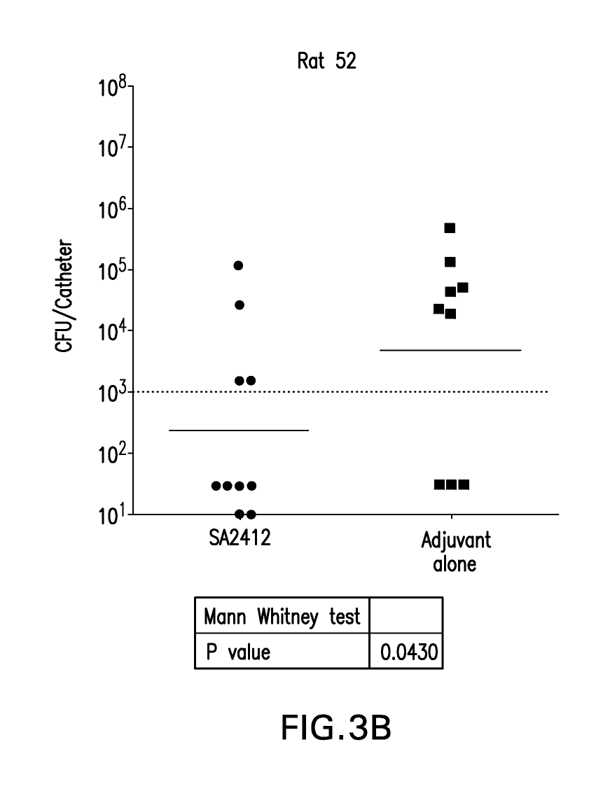

After two independent experiments, 7 of 17 catheters were culture positive (41%) in rats immunized with His-tagged SA2412, whereas, 12 of 18 catheters were culture positive in the control rats (67%). The results are shown in Table 1. FIGS. 3A-3B show the CPUs measured on the catheters collected from the rats. The average number of CFUs isolated from the negative control rats was higher than the average number of CFUs measured on the catheters from rats vaccinated with His-tagged SA2412 (combined p value=0.07).

TABLE 1

| | Number of culture | | |
|---|---|---|---|
| | positive catheters | | Total number |
| | Experiment | Experiment | of culture positive |
| Treatment | #1 | #2 | catheters (%) |
| His-tagged SA2412 + | 3/7 | 4/10 | 7/17 (41%) |
| adjuvant | | | |
| adjuvant alone | 6/9 | 6/9 | 12/18 (67%) |

### Example 4

### Protection Studies Using a Murine Lethal Challenge Model

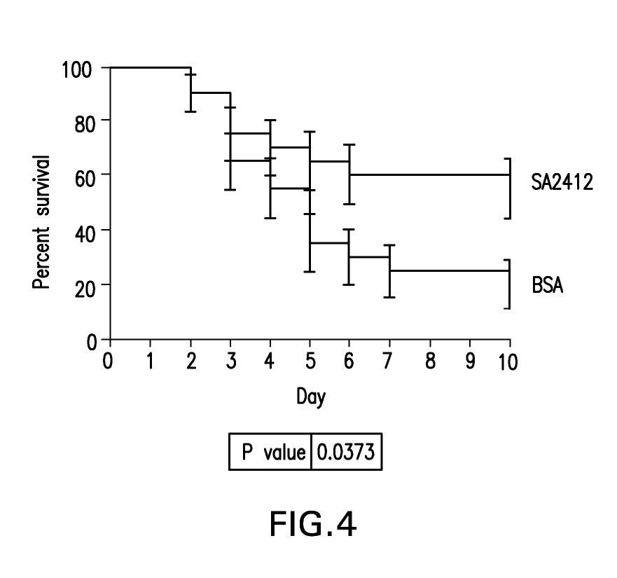

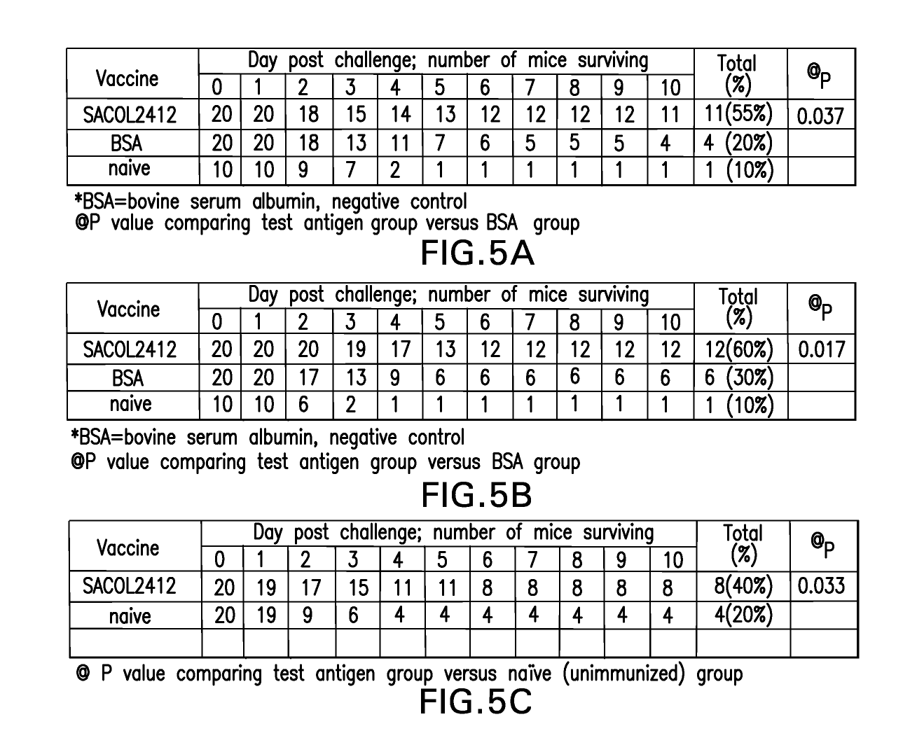

In three independent experiments, Balb/c mice immunized with either SA2412 or BSA formulated on AAHSA (20 per group) or unimmunized (see EXAMPLE 2) were challenged with S. aureus Becker (8×10⁸ CFU/mouse) injected via the tail vein two weeks after immunization. Mice were monitored for survival for a period of 10 days post challenge. At the end of the first experiment, 11 mice survived in the SA2412 polypeptide-immunized group, compared to 4 surviving in the BSA control group. Thus, the group of mice immunized with SA2412 had a significantly enhanced survival rate compared to the negative control mice immunized with BSA (p=0.0373). Results are shown in FIG. 5A and survival curves are compared in FIG. 4. In the second experiment, 12 mice survived in the SA2412 polypeptide-immunized group, compared to 6 surviving in the BSA control group. The results are shown in FIG. 5B. Similar to the first experiment, mice immunized with SA2412 had a significantly enhanced survival rate compared to the negative control mice immunized with BSA (p=0.017).

In a third independent experiment, 20 Balb/c mice were immunized intramuscularly with 20 μg of His-tagged SA2412 adsorbed onto AAHSA on days 0, 7, and 21 and compared to a similar group of naïve (unimmunized) mice. Two weeks later, mice were challenged by intravenous injection of S. aureus strain Becker (dose 8×10⁸ CFU/mL) and monitored for survival for a period of 10 days. At the end of this experiment, 8 mice in the SA2412-immunized group survived compared to 4 in the naïve (unimmunized) group (p=0.033). Results are shown in FIG. 5C.

Other embodiments are within the following claims. While several embodiments have been shown and described, various modifications may be made without departing from the spirit and scope of the present invention.

1. A composition comprising an immunologically effective amount of a polypeptide that is at least 95% identical to SEQ ID NO:1 or a fragment of the polypeptide and a pharmaceutically acceptable carrier.

2. The composition of claim 1 wherein the polypeptide is SEQ ID NO:1.

3. The composition of claim 1 further comprising one or more additional S. aureus antigens.

4. (canceled)

5. The composition of claim 1 wherein the composition further comprises an adjuvant.

6. A method of inducing a protective immune response in a patient against an S. aureus infection comprising the steps of administering to the patient an immunologically effective amount of the composition of claim 1.

7. The method of claim 6 wherein the patient is human or a non-human mammal.

8. The method of claim 7 wherein the patient has weakened immunity, has received a foreign body implant or is on renal dialysis.

9. The method of claim 8 wherein the patient that weakened immunity has HIV or AIDS.

10. The method of claim 8 wherein the foreign body implant is a catheter, a vascular device, pacemaker leads, defibrillator systems, or prosthetic heart valve.

11-17. (canceled)

18. A method of conferring passive immunity to S. aureus infection in a patient comprising administering to the patient one or more antibodies that specifically bind to a polypeptide of SEQ ID NO:1.

19. The method of claim 17 wherein the one or more antibodies are monoclonal antibodies.

20. The method of claim 18 wherein the one or more antibodies are selected from the group consisting of human antibodies and humanized antibodies.


SEQUENCE LISTING (3 sequences)

SEQ ID NO: 1 - protein, 259 aa, S. aureus
MKRLLFVMIAFVFILAACGNNSSKDKEASKDSKTINVGTEGTYAPFSFHDKDGKLTGYDIDVIKAVAKEEGLKLKFNETSWDSMFAGLDAGRFDVIANQVGINPDREKKYKFSKPYTFSSAVLVIRENEKDIKDFDDVKGKKLAQTFTSNYGKLAKDKGADITKVDGFNQSMDLLLSKRVDGTFNDSLSYLDYKKQKPNAKIKAIKGNAEQSRSAFAFSKKADDETVQKFNDGLKKIEENGELAKIGKKWFGQDVSKSK

SEQ ID NO: 2 - DNA, 780 bp, S. aureus
ctatttagatttagaaacatcttgaccaaaccatttcttacctattttagctaattcaccgttttcctcgatttttttcaagccatcattgaatttttgaactgtttcatcatctgcttttttagaaaatgcaaatgcagatctactttgttcagcattacctttgattgctttgatcttagcattaggtttttgttttttataatccaagtatgacagactatcattaaatgtaccatcaacacgcttagacaataataaatccattgattggttaaagccatcaacttttgtaatatcagcacctttatccttagctaatttaccataattagatgtgaatgtttgtgctaacttcttacctttaacatcatcaaaatctttaatatctttttcattttcacgaataactaaaacagcacttgagaatgtgtaaggcttagaaaatttatatttcttttctctatcaggattaatacctacttggttcgcgattacatcaaaacgccctgcgtctaaacctgcaaacatagaatcccaagaagtttcattaaatttaagttttaaaccttcttctttagccactgctttaataacatcaatatcgtaaccagttaatttaccatctttatcgtggaaactaaatggtgcataagtcccctcagtcccaacattaattgtcttgctatctttactagcttccttgtctttcgacgaattgtttccgcatgcagccaatatgaaaacgaaagctatcatcacaaataaaagtcttttcat

SEQ ID NO: 3 - protein, 645 aa, S. aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN